(19) United States
(12) Patent Application Publication
(10) Pub. No.: US 2020/0206961 A1
(43) Pub. Date: Jul. 2, 2020

(54) BACKDRIVABLE AND HAPTIC FEEDBACK CAPABLE ROBOTIC FORCEPS, CONTROL SYSTEM AND METHOD

(71) Applicant: Ugur TÜMERDEM, Istanbul (TR)

(72) Inventor: Ugur TÜMERDEM, Istanbul (TR)

(21) Appl. No.: 16/612,658

(22) PCT Filed: Aug. 8, 2017
(86) PCT No.: PCT/TR2017/050374

§ 371 Date: Nov. 11, 2019

(51) Int. Cl. B25J 17/02; A61B 34/00; A61B 34/37; B25J 3/04

(52) U.S. Cl. CPC B25J 17/0266; A61B 34/76; A61B 2034/304; B25J 3/04; A61B 34/37

(57) ABSTRACT

Disclosed is a highly backdrivable robotic forceps that can have up to 7 degrees of freedom (DOF) similar to the human wrist/hand and that enables 7 DOF force estimation for use in minimal invasive robotic surgical systems and a robotic forceps control system and method allowing force feedback teleoperation (bilateral) of said robotic forceps. The robotic forceps mechanism is a structure capable of bilaterally controlled motion and having the capability to mimic the hand movements of a surgeon and reflection of the forces on the forceps tip to the surgeon's control interface. Control and estimation of forces applied on the forceps tip can be achieved thanks to the novel backdrivable structure of the robotic forceps mechanism and the control system and method presented here.

### THE RELATED ART

The invention relates to a backdrivable robotic forceps mechanism that can have up to 7 degrees of freedom (DOF) similar to the human wrist/hand and that enables 7 DOF force estimation for use in minimal invasive robotic surgical systems and a robotic forceps control system and method providing force/haptic feedback remote control/teleoperation of mechanisms having similar backdrivability characteristics.

### THE PRIOR ART

One of the methods for performing operations with minimal damage to a patient is to conduct operations in a patient's body by means of entering the body through 0.5-1.5 cm ports/incisions by use of remotely controlled cameras and special laparoscopic forceps instruments. This method is also called minimal invasive surgery (MIS) or laparoscopic surgery. Use of robotic systems in MIS operations has become popular in the past 20 years due to mainly ergonomic problems for the surgeons in minimal invasive surgical operations. The most prominent example of such robotic surgery systems is the Da Vinci Surgical System by Intuitive Surgical. In robotic minimal invasive surgery, surgeon sits at a console, moves 2 robotic control arms with his/her hands and provides movement and remote operation of robotic forceps instruments inserted into patient's body through incisions/ports opened on the patient body and having the same degrees of freedom as the human hand (7 degrees of freedom assuming grasping is a single degree of freedom task). With this method, the 2 bending degrees of freedom of the human wrist that are often lost in conventional laporoscopic surgery can also be utilized by the robotic forceps inside the patient's body, by means of a remotely controlled wrist mechanism which is found at the tip of each robot arm. Furthermore, surgeons can perform operations more comfortably, faster and with higher precision, as they will perform the operation while seated, and the instruments can move in the same directions as the surgeon's hands, and the hand movements can be scaled down and vibrations are filtered. The biggest disadvantage of this method in comparison to conventional laparoscopic surgery is surgeon's lack of tactile information about the surfaces where the instrument touches, since there is no feedback of the measured forces/torques applied on the forceps via the user interface (haptic feedback).

Surgeons using robotic surgery systems perform operations without the sense of touch, and therefore cannot perform operations efficiently. One of the most important reasons thereof is that during operation, the intracorporeally (inside the body) located robotic forceps wrists-grippers are controlled by cable pulley mechanisms actuated extracorporeally (from outside the body) in order to perform 90 degree pitch-yaw motions (See FIG. 1) and jaw opening-closing (gripping) motions. Today, Da Vinci surgical robots make use of 3 DOF (or less) intracorporeally utilized wrist mechanisms attached to extracorporeal robotic arms which increase the total degrees of freedom of the robotic forceps to 7. The motions of the wrist and the gripper operating inside the body are controlled by cable pulley mechanisms, and the other degrees of freedom are controlled by backdrivable robot arms which are located outside the body.

The forces/torques generated on the instrument tip cannot be fully transmitted to extracorporeal motors or sensors due to friction, slips and slacks and other nonlinearities on cable-pulley systems which result in a loss of back-drivability. In addition, force/torque sensors which can pass through incisions of 1.5 cm or less and can be used in a patient's body have to be very small. Production of such sensors is very difficult and expensive. Although not commercialized, the only system having the potential to achieve force/torque measurement at 7 degrees of freedom in patient's body and transmit the measurements to the surgeons is the experimental system of DLR called “MIRO Surge”. In order to perform this, the smallest force/torque sensor in the world with 6 degrees of freedom has been developed and mounted on this system. The biggest disadvantage of the system is that the control of wrist motions is provided by means of cable-pulley mechanism again and that the wrist has rotation capacity that is half that of the Da Vinci system (45 degrees bending). Therefore, robotic forceps systems capable of estimating and controlling 2 or 3 degrees of freedoms (pitch, yaw, gripping) intracorporeally and also having the large workspace and mobility of the commercially available robotic surgery systems (Da Vinci) are not encountered in the prior art.

### BRIEF DESCRIPTION OF THE INVENTION

A purpose of the invention is to provide a robotic forceps wrist-gripper mechanism for intracorporeal use, which is
capable of jaw opening-closing (gripping) motion to grasp or cut an object,
capable of passing through a small incision and intracorporeally conducting bending wrist motions up to 90 degrees (90 degrees in each direction from starting pose, 180 degrees total) around two perpendicular axes (pitch, yaw) within the patient's body,
capable of force estimation in three degrees of freedom (pitch, yaw bending and gripping) at the same time in the intracorporeal section of the forceps without the use of a force sensor
controlled by extracorporeal actuators through rigid rods which are capable of transmitting the forces and torques acting on the forceps instrument, to actuators enabling the estimation of the force/torques through actuator measurements,
capable of precisely controlling the position/orientation of the forceps, and the forces/torques applied by the forceps on the surfaces it touches, according to commands by a remote operator/user, and capable of reflecting the forces/torques applied on the forceps to the operator by means of a command interface.

The second purpose of the invention is to provide a force estimation and control method for the mechanism disclosed herein, and mechanisms that operate through a similar principle. In the present application and in robotic literature, force/torque control is used as synonyms in several cases. For instance, force control for roll-pitch-yaw rotation degrees of freedom means the control of torque applied on the mentioned degrees of freedom. The wrist-gripper mechanism according to the invention can move in 7 degrees of freedom by being mounted to a robotic mechanism found outside the body and force estimation and control will also be applied on this external mechanism so as to ensure force estimation and control in all 7 axes. The control and force estimation method according to the invention is also applicable for wrist-gripper mechanisms which have similar features to the wrist-gripper mechanism disclosed herein such as backdrivability or mechanisms which utilize similar principles (extracorporeal actuation via rigid rods as transmission) for operation.

The most important novelty of the 3 degrees of freedom robotic forceps wrist/gripper mechanism disclosed hereunder and forming example for the invention is that wrist bending motions (pitch-yaw) of 90 degrees and opening-closing motions are controlled by means of coordinated motion of 3 rigid rods and 3 linear motors without the use of cable and pulley system. This approach also enables back-drivability of the system. In other words, just like the forceps moves as a result of the motion of the motors, the motors go through exactly the same motion when the forceps is moved by externally applied forces in the same manner.

In the related art, wrist mechanisms capable of providing bending motions of 90 degrees in one direction (180 degrees in two directions) in two axes and moved by rigid rods are used in other applications but are not used in robotic surgery. In addition, there is no mechanism capable of bending 90 degrees in 2 axes while conducting jaw opening closing (gripping) motions. The robotic forceps mechanisms using linear transmission rods have considerably limited wrist roll motion or can conduct 90 degrees rolling motions only in one axis. In addition to these, the systems disclosed in Reboulet (1992), Yamashita et. al. (2005), Merlet (2002), Wallace et. al. (2006), Peirs et. al. (2000), Salle et. al. (2004), Dohi et. al. (2004), Nakamura et. al. (2004), Burbank (2014), Choi ve Kim (2012), Grace (2000) can also be given as examples. These systems have between 2-4 degrees of freedom. They can make wrist bending/rotations at 30-90 degrees range. However, they are not capable of performing 90 degrees rotations in both pitch and yaw axes. Furthermore, taking into account the back-drivability, force estimation technique has not been achieved by these systems. Only Arata et al. (2005) has developed a robotic endoscope capable of 3 degrees of freedom (pitch, yaw and jaw opening-closing) motion by use of push rods, and this system has an extra corporal force sensor located between rod conducting jaw opening-closing motion and motor, and has force feedback. This system is capable of achieving force feedback only in one (gripping) axis.

However, in our invention, the forces applied on the wrist are exactly transmitted to actuators/motors via rigid rods, thus it is possible to estimate forces by means of running algorithms controlling the servo motors. This estimation cannot be achieved easily when there is a lack of a rigid connection between the actuators and the wrist, such as in the systems using cable-pulley mechanisms. In wrist mechanisms with rigid transmission, force estimation has not been achieved because of a lack of algorithms providing force estimation at all degrees of freedom via actuator measurements making use of the back-drivability feature. For that reason, the invention can enable wrist motions of 90 degrees in 2 perpendicular axes (pitch, yaw), gripper opening-closing motion and also enable accurate estimation and control of instrument forces on the mentioned axes without the use of a force sensor. The force estimation and control method as presented by the invention can be used in conjunction with the mechanism disclosed with the invention as well as back-drivable mechanisms, working with a similar principle such as mechanisms utilizing rigid transmission rods. The control method can be partially adapted to cable-pulley mechanisms or mechanisms with lower backdrivability, but in that case there would be a loss of force estimation accuracy and fidelity.

For better understanding of the embodiment of the present invention and its advantages with its additional components, it should be evaluated together with below described figures.

### BRIEF DESCRIPTION OF THE FIGURES

FIG. 1 is general view of robotic forceps wrist-gripper mechanism according to the invention.

FIGS. 2, 3 and 4 are views of gripper part, wrist motion mechanism and driving mechanisms parts of an embodiment of the robotic forceps mechanism disclosed herein.

FIG. 2a is a detailed view of the pin-hole mechanism utilized by the grasping element of an embodiment of the robotic forceps mechanism in the invention.

FIG. 5 is a detailed view of sections of robotic forceps mechanism according to the invention.

FIG. 6 is the view showing the installation of an embodiment of the robotic forceps wrist gripper mechanism according to the invention, to a robot arm.

FIG. 7 is the view showing bilateral tele-operation data communication between robotic forceps control computer and master control computer.

FIG. 8 is the view showing data communication between robotic forceps control computer and robotic forceps mechanism.

FIG. 9 shows flow diagram of a version of the bilateral teleoperation algorithm.

FIG. 10 shows flow diagram of an alternative version of bilateral teleoperation algorithm.

FIG. 11 shows flow diagram of force estimation algorithm.

### REFERENCE NUMBERS

A Gripper section
1 Gripper
3 Gripper base
4 Pin-slot mechanism
4.1 Pin section
4.2 Slot section
B Wrist motion mechanism
5 Upper connection part
6 Primary mid-connection part
7 Secondary mid-connection part
8 Lower connection part
9 Mechanism base
10 Height fixing columns
11 Height fixing column primary joint
12 Height fixing column secondary joint
C Actuator transmission section
13 Interconnection piece
14 Motion transmission rods
15 Primary base column
16 Shaft bearing
17 Secondary base column
D Driving mechanism
19 Connection rods
20 Shaft
21 Forceps motor
22 Fixer member
23 Robotic forceps base
24 Rotary motor
25 Robot arm connection apparatus
26 Robot arm
27 Arm base
28 Forceps DAQ card and motor drivers
29 Robotic forceps mechanism
30 Master control interface
31 Robotic forceps control computer
32 Kinematic transformation
33 Inverse dynamics calculation
34 Position controller
35 Disturbance estimator
36 External force estimator
37 Speed estimator
38 Damping
39 Master control computer
40 Band pass filter
41 Master motors
42 Motor model (transfer function)
43 Motor inverse model (inverse transfer function)
44 Master DAQ card and motor drivers

### DETAILED DESCRIPTION OF THE INVENTION

In this detailed description, the novelty according to the invention is only disclosed for better understanding of the subject without forming any limiting effect.

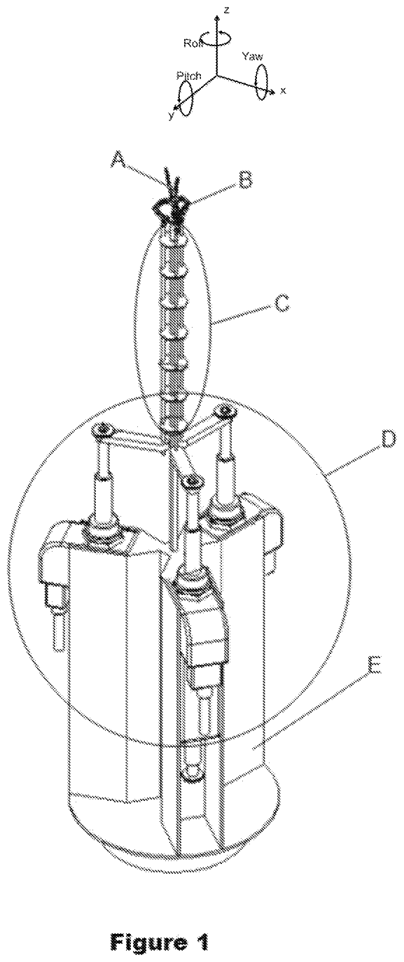

The robotic forceps according to the invention is articulated to a robotic mechanism that can enter into human body through a 1.5 cm port, and then allows performing the hand movements of the surgeon in 7 degrees of freedom within the patient body (intracorporeally), one to one or in a scaled-down manner. The diameter of port to be used for entering the patient's body can be reduced as much as the wrist mechanism dimensions can be reduced. The motions required from human hand and thus a robotic forceps are translation in x, y, and z axes; rotation in roll, pitch, and yaw axes, and gripper opening and closing movements that correspond to opening and closing of the hand. (See FIG. 1)

The robotic forceps system according to the invention comprises a robotic forceps mechanism (29) which is articulated to a robotic mechanism and can be used for force estimation and control in all 7 degrees of freedom, and will have motion in at least 1 degree of freedom (gripper, pitch, or yaw) in the body. FIG. 1 shows general view of the robotic forceps mechanism (29) with 3 degrees of freedom, which may be exemplary for the present invention. The robotic forceps mechanism (29) mainly comprises:
a gripper section (A) performing the gripping/grasping and cutting operations;
a wrist motion mechanism (B) enabling the desired rotational wrist motions (such as pitch and yaw) and the gripper opening/closing motion of the said gripper section (A),
a actuator/driving mechanism (D) having motors (21) providing the required drive for the motion of said wrist mechanism (B) and capable of conducting position and force control by means of various control algorithms,
an actuator transmission section (C) transmitting the motion provided by the forceps motors (21) to the wrist motion mechanism (B), and transmitting the force generated on the wrist motion mechanism (B) to the forceps motors (21), and
a pedestal section (E) on which the system components are mounted.

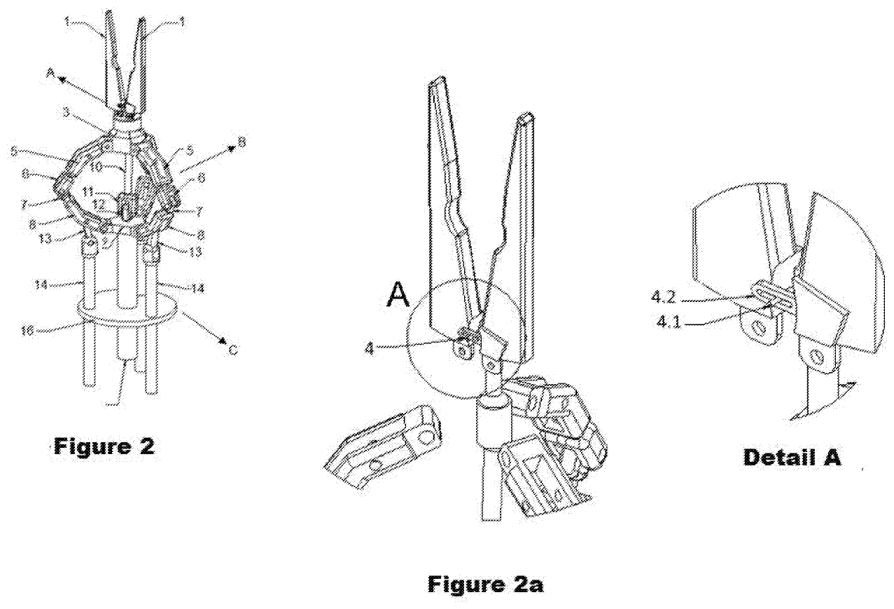

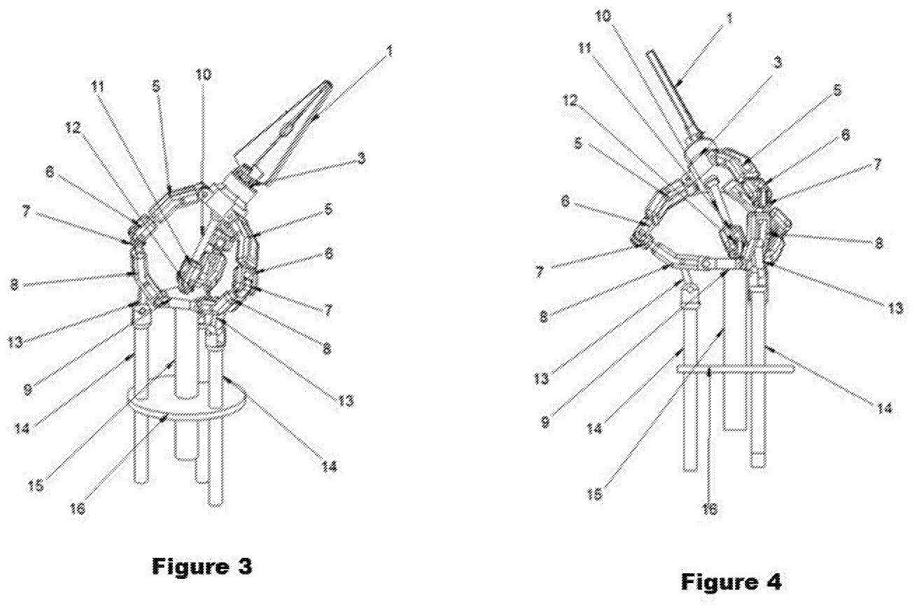

FIGS. 2, 3, and 4 show said gripper section (A), wrist motion mechanism (B), and driving mechanism (D).

The gripper section (A) comprises:
a gripper (1) performing the gripping/grasping and cutting operations;
a gripper base (3) on which each jaw of said gripper (1) is mounted by means of revolute joints, and which constitutes the connection point of the gripper (1) and the wrist mechanism (B),
a pin-slot mechanism (4), the pin section (4.1) of which is fixed to height fixing columns (10), the slot section (4.2) of which is found on the gripper (1), and which ensures conversion of the relative linear motion between the height fixing columns (10) and the gripper (1) into rotational motion around the rotary joints where the gripper jaws are connected on the gripper base, and thus ensures conversion into the gripper opening-and-closing motion,
a gripper base (3) forming the ceiling of the wrist motion mechanism (B) and the base of the gripper section (A), determining the general orientation/motion of the gripper (1), and providing relative motion of the slot (4.2) with regard to the pin (4.1).

The wrist motion mechanism (B) comprises:
upper connection pieces (5) connected to the wrist mechanism ceiling, which is also the gripper base (3), through revolute joints, and ensuring movement of the gripper (1) too, when the lower part of the mechanism is moved, and transmitting the forces generated at the gripper (1) to the other parts of the mechanism,
primary mid-connection parts (6) connected to the said upper connection parts (5) with revolute joints and allowing the gripper (1) to change its orientation via rotation on a single axis,
secondary mid-connection parts (7) connected to the said primary mid-connection parts with revolute joints (6) and allowing the gripper (1) to change its orientation by means of rotating on a second axis that is perpendicular to the part to which it is connected,
lower connection parts (8) connected to the said secondary mid-connection parts (7) with revolute joints and providing transmission of the motion coming from the forceps motors (21) to the gripper base (3) and the forces coming from the gripper base (3) to the forceps motors (21),
a mechanism base (9) serving as the base of the mechanism, to which the lower connecting parts (8) are connected and all static forces on the mechanism are transmitted,
height fixing columns (10), composed of two or three columns connected to each other with spherical joints or similar, preventing back-and-forth motion of the pin section (4.1) in the pin-slot mechanism (4), and thus providing relative motion of the gripper base (3) and the slot section (4.2) with respect to the pin section (4.1),
a height fixing column primary joint (11) to which the height fixing columns (10) are connected and which also allows the height fixing columns (10) to change their orientation in accordance with the rotation of the gripper base (3),
a height fixing column secondary joint (12) to which said height fixing column primary joint (11) is connected and which allows the primary joint to also change its orientation in an axis perpendicular to the primary joint axis.

The purpose of using the height fixing columns (10) and joints (11, 12) is to keep the radial length of the pin (4.1), found in the pin-slot mechanism (4) on the gripper (1), fixed with regard to the mechanism base (9). Thus, when the radial motions of the gripper (1) (defined on the axis connecting the centers of the mechanism base (9) and gripper base (3)) are controlled by the wrist motion mechanism (B), gripper (1) opening-closing motion can be provided by means of the pin-slot mechanism (4). Height fixing columns and joints can be substituted by different types of joints and columns such as semi-rigid links providing resistance against forces in radial direction while being capable of bending, or non-rigid flexible string like materials. Similarly, the pin-slot mechanism can be substituted by other similar mechanisms (i.e. 4-bar mechanism etc.) capable of converting the relative linear motion into rotary gripper opening-closing motion. Also, different joint types (such as ball joints) that would allow similar mechanism function can be used instead of the rotary joints in the connection parts.

The actuator transmission part (C) is the component which basically provides transmission of motion/force between the wrist motion mechanism (B) and the forceps motors (21), and it comprises:
interconnection pieces (13) connected to the lower connection parts (8) and the motion transmission rods (14) via rotary joints where the interconnection pieces (13) intersect with both the lower connection parts (8) and the motion transmission rods (14) and providing rotation of the lower connection part (8) to which it is connected, depending on the motion received from the forceps motors (21), and thus indirectly allowing the gripper base (3) to change its orientation,
motion transmission rods (14) connected to said interconnection parts (13) with revolute joints and transmitting the motion received from the forceps motors (21) to the wrist motion mechanism (B), and also allowing transmission of the forces and motions formed at the gripper (1) to the forceps motors (21) without any loss,
a primary base column (15) and a secondary base column (17) extending parallel to the said motion transmission rods (14) along the actuator transmission part (C) and supporting the mechanism base (9),
shaft bearings (16) having openings where motion transmission rods (14) can be inserted through and preventing out-of-axis motions of the motion transmission rods (14) placed in these openings, and reducing the radial loads thereon.

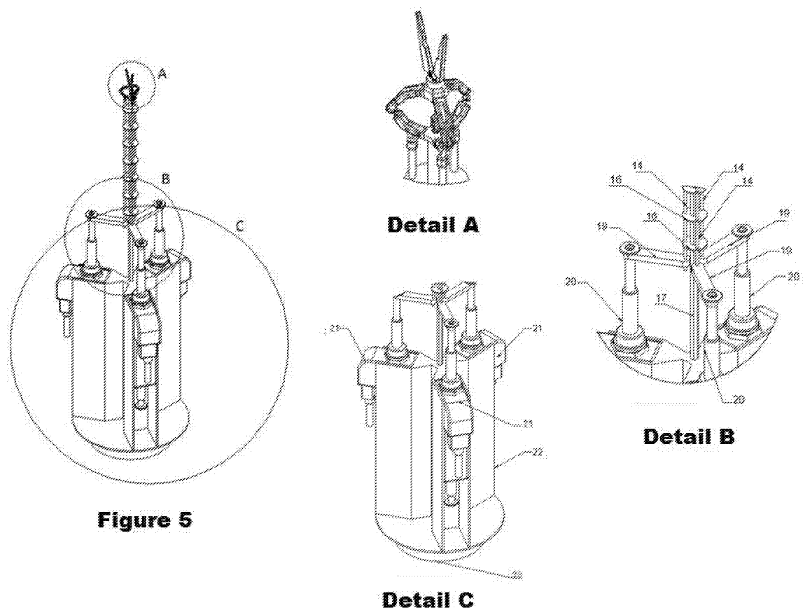

FIG. 5 shows detailed views of the parts of the robotic forceps mechanism (29). In the figure, the driving mechanism (D) and the relationship of the driving mechanism (D) with the actuator transmission part (C) are also shown. Accordingly, the driving mechanism (D) comprises:
forceps motors (21) capable of performing force and position control by means of control algorithms,
shafts (20) which are the movable parts of the forceps motors (21),
connection rods (19), at one end of which the motion transmission rod (14) and at the other end of which the shaft (20) are connected so as to provide connection between the motion transmission rods (14) and the forceps motors (21).

The said forceps motor (21) is preferably a linear motor. Different driving mechanisms such as rotary motors, pneumatic actuators, or hydraulic actuators can also be used with minor modifications. The base part (E) comprises fixing members (22) aligning the forceps motors (21) relative to one another and fixing thereof to the base of the robotic forceps mechanism (29).

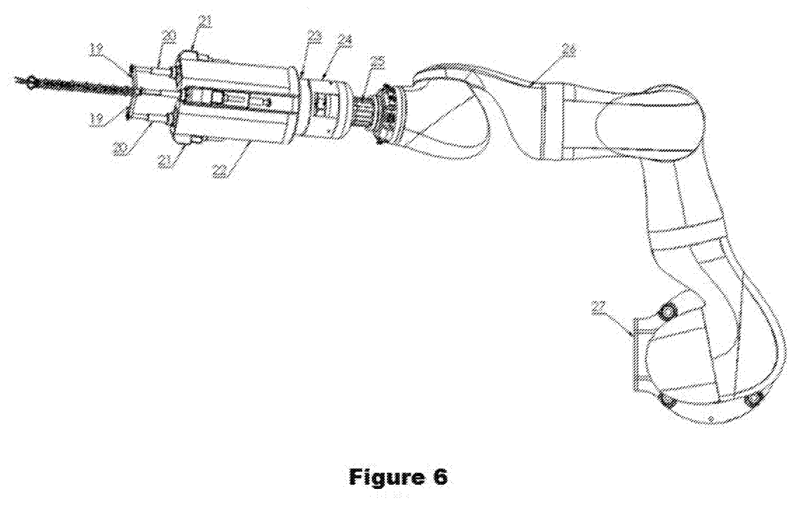

FIG. 6 shows a robotic forceps mechanism (29) as mounted to a robot arm (26). A rotary motor (24) is positioned at the base (23) of the robotic forceps mechanism (29) so as to provide an extra degree of freedom to the system and to ensure that the whole forceps mechanism rotates (rolling motion) around z axis, which is the direction of the linear motors' motion. This increases the total degrees of freedom of the wrist mechanism to 4 (roll, pitch, yaw, gripper's opening-closing motions can be made by the forceps). The robot arm (26) enables the robotic forceps mechanism (29) having 3 degrees of freedom to reach 7 degrees of freedom together with the rotary motor (24) (roll, pitch, yaw, opening closing, x, y, z motions can be conducted). The duty of the rotary motor (24) can also be performed by the robot arm (26). The robot arm (26) should be able to allow force estimation and be back-drivable. The robotic forceps mechanism (29) is connected to the robot arm (26) by a connection apparatus (25). The connection apparatus (25) is connected to any robot arm (26) and enables increasing the degrees of freedom as desired. While the robot arm (26) can be a system that can be purchased in ready-made form, it can also be formed of backdrivable mechanisms with fewer axes that can be custom-designed for the operation.

Forceps motors (21) allow the wrist motion mechanism (B) to make rotating motions (pitch and yaw) around x and y axes and the gripper base (3) to make back-and-forth motions on radial axis. Radial axis is located on the line between the centre of the gripper base (3) and the centre of the mechanism base (9), and changes its direction as the wrist (B) rotates. The back-and-forth motion on the radial axis is converted into gripper opening-closing motion via the pin-slot mechanism (4) located on the gripper (1). The shafts (20) are connected to the motion transmission rods (14) via the connection rod (19) and provide motion of the wrist motion mechanism (B) by means of rotation of the lower connection part (8) with the help of the interconnection piece (13). The mechanism base (9) acts as the pedestal of the mechanism and all the loads on the mechanism are transmitted to this part. The primary and secondary base columns (15, 17) used for supporting the mechanism base (9) also connect the mechanism base (9) to the robotic forceps base (23). Shaft bearings (16) prevent the out-of-axis motion of the motion transmission rods (14) and thus reduce the loads thereon. Motion transmission rods (14) are connected to the lower connection parts (8) of the wrist motion mechanism (B) via the interconnection parts (13). With the collective motions of the mid-connection parts (6,7) connected to one another and the upper (5) and lower connection parts (8) with revolute joints, the linear motions of the transmission rods (14) are converted into wrist mechanism (B) rotation and radial motions. In this way, the gripper base (3) is ensured to achieve the required (pitch, yaw) orientation and gripper can perform the jaw opening-closing motions. The parts forming the wrist motion mechanism (B) are also interconnected via revolute joints. The lower connection rod (8) ensures the motion and the force transmission between the forceps motors (21) and the gripper base (3). The mid-connection parts (6, 7) rotate in axes that are perpendicular to the upper connection parts (5) and the lower connection parts (8). Mid-connection parts (6, 7) are also connected to one another via revolute joints. This implies that the mid-connection parts (6, 7) together behave like a spherical joint. Thanks to the symmetrical structure of the wrist motion mechanism (B) arms, the lower and upper connection parts (5, 8) rotate in the same amount.

The height fixing columns (10) passing through the gripper base (3) and connecting the pin-slot mechanism (4) found on the gripper with the mechanism base (9) can adjust the radial distance of the pin (4.1) found on the gripper with regard to the mechanism base (9). The height fixing columns (10), connected to the gripper base (3) via ball joints or two different joints with axes perpendicular to each other, rotate together with the wrist during rotation of the wrist so as to prevent radial motion of the pin section (4.1) of the opening-closing mechanism (4), and thus allow relative motion between the slot section (4.2) and the pin. The gripper (1) moving by means of the gripper base (3) around these columns causes relative motion between the pin (4.1) and the slot (4.2) and allows opening-closing of the gripper (1) jaws via this mechanism. The reason for using of two perpendicular joints (11, 12) or ball joint at the base of the height fixing columns (10) is to ensure that the pin (4.1) and slot (4.2) found on the gripper opening-closing mechanism (4) maintain the same orientation with each other and thus avoid restriction of motion, when the wrist motion mechanism (B) is oriented in various angles. The primary joint (11) of the height fixing columns allows rotation of the height fixing columns (10) and thus columns do not prevent motion as the gripper base (3) rotates. The height fixing column secondary joint (12) enables a similar rotation perpendicular to column primary joint (11). A single ball joint can be used instead of these two joints.

The opening-closing mechanism (4) performing the gripper (1) jaw opening closing motion converts the relative displacement motion between the mechanism base (9) and the gripper base (3) into rotational opening-closing motion. While the design disclosed herein comprises a pin-slot mechanism (4), any other mechanism (such as 4-bar mechanism, or a gear set) that can convert the relative linear motion into rotational motion may also be used.

In an alternative embodiment of the robotic forceps mechanism (29) according to the invention, the gripper section is found as a separate module. In this module, the motion of a rotational micro motor is converted into a gripper opening-closing motion by means of a worm gear. In another alternative, the gripper comprises a linear micro motor and the back-and-forth motor motions are directly converted into gripper motion with a 4-bar like mechanism. In both alternatives, since both the gripper (1) and the wrist motion mechanism are rigidly connected to motors, the forces on these axes are transmitted to the motors, and the sizes and directions of these forces are determined by means of estimation algorithms making use of real time data obtained from the motors. Since, in this alternative design, the motion of the wrist mechanism (B) in radial axis is not converted into gripper opening-closing motion, this radial motion by the wrist mechanism can be utilized as the thrust motion of the forceps. In another version of the invention, the gripper opening-closing motion can be performed though extracorporeal actuators by means of a cable pulley system, but in this case, force estimation and control at the gripper axis becomes more difficult due to mentioned problems in such systems.

The robotic forceps control system comprises a master control interface (30) so as to provide remote control of the robotic forceps mechanism (29) by an operator. The robotic forceps mechanism (29) to be employed in the system needs to have at least 1 degree of freedom in body. Other required degrees of freedom can be provided by means of a mechanism that is outside the body, and the control system can also be applied in the same way on a system having up to 7 degrees of freedom. In such a case, it is not needed to change the control method steps, but only, the method steps/parameters to be used need to take into account the dynamic/kinematic parameters of the external mechanism to which the forceps mechanism is mounted.

The master control interface (30) is the unit controlled by the operator manually and thus sensing the position of the operator's hand and the force applied by the hand. The master control interface (30) should have the same degrees of freedom with the number of degrees of freedom of the robotic forceps to be controlled. At the same time, it should also have actuators capable of reflecting/applying force onto the surgeon hand, and so it is a robotic system. The master control interface (30) is connected to a master control computer (39). The master control computer (39) communicates with a robotic forceps control computer (31) to which the robotic forceps (29) is connected.

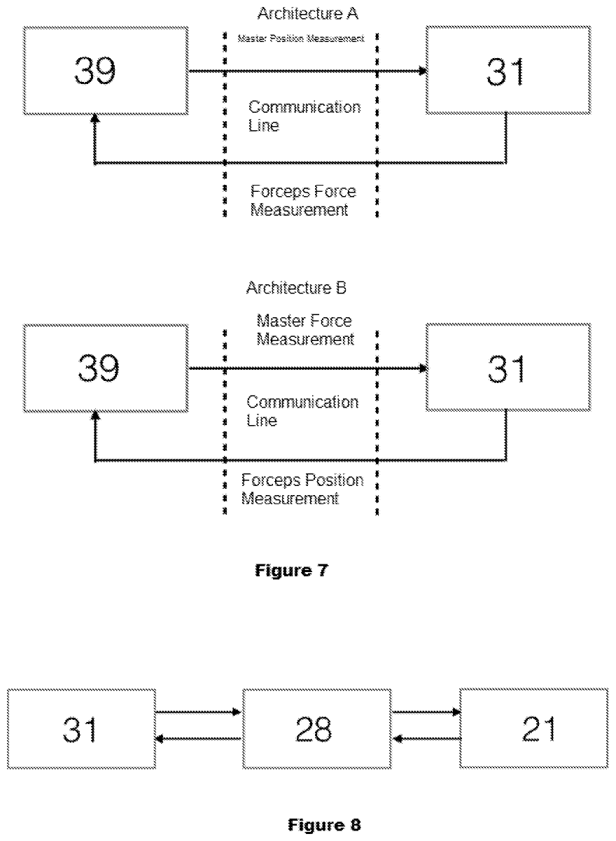

FIG. 7 shows two alternatives of bilateral (two-way) teleoperation data communication between the robotic forceps control computer (31) and the master control computer (39). In said both alternatives, one of the robots works in force control mode while the other one operates in position control mode (Architecture A). In the other architecture (Architecture B), the robots by which force control and position control are made are interchanged. In order to bring the robotic forceps (29) to a desired position by the master control interface (30), one of the robots needs to be operated under position control; and in order to feel the forces applied onto the robotic forceps (29) through the master control interface (30), the other robot needs to work in force control mode. The robot working in position control mode takes the position data of the other robot as a reference signal and a setpoint, while the robot working in force control mode takes the force estimation data measured on the other robot as reference.

FIG. 8 shows signal/data flow between the robotic forceps control computer (31) and the robotic forceps motors (21) and between the master control computer (39) and the master motors (41). The robotic forceps control computer (31) processes the reference force or position information received from the master control computer (39) by means of the control and force estimation algorithms comprised therein, and thus sends signals to the motors (21) of the robotic forceps mechanism (29) so as to allow them to perform the determined task (force or position control). The master control computer (39) processes the reference force or position information received from the robotic forceps control computer (31) by means of the control and force estimation algorithms comprised therein, and thus sends signals to the master motors (41) so as to allow them to perform the determined task. DAQ cards and motors drivers (28, 44) are utilized to ensure communication between the computers (31, 39) and the motors (21, 41). DAQ/Signal processing card and motor drivers (28, 44) are utilized to read signals from motor encoders and provide the computers with motor position data and transmit current commands to the motors. The drivers control currents and thus motions of the motors (21, 41) according to the digital or analogue commands from the DAQ cards. Similarly, they receive the current and position data of the motors (21, 41) and ensure transmission of the same to control computers (31, 39) through the DAQ card.

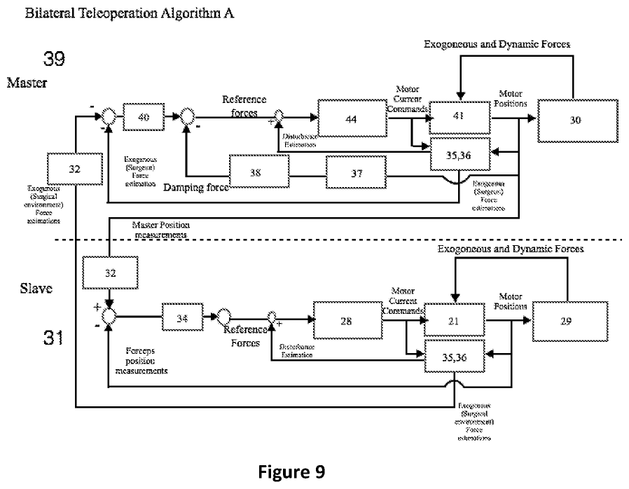

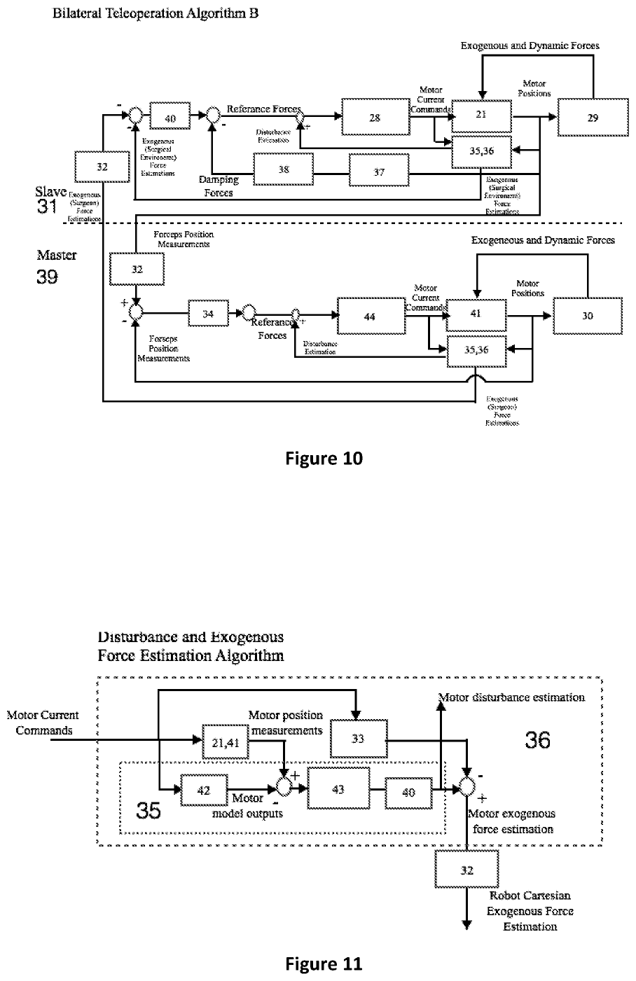

FIGS. 9 and 10 show detailed flow diagrams of the two alternatives of the bilateral teleoperation algorithm that is to run on the master control computer (39) and the robotic forceps control system (29). During bilateral tele-operation, remote control of the robotic forceps (29) and feeling of the force feedback by the surgeon is ensured by means of control algorithms running simultaneously in the master control computer (39) and the forceps control computer (31). In the case that the master control interface (30) and the forceps systems are within the same location, the master control computer (39) and the forceps control computer (31) can be a single computer and the algorithms can run on a single computer.

According to FIG. 9, the control and force estimation method (Architecture A) of the bilateral teleoperation controller working simultaneously in the master control computer (39) and the robotic forceps computer (31) comprises following process steps. The method foresees repetition of the steps several times per second, by the control system, as a loop.
Feeding of the force estimation signals, which are transmitted from the robotic forceps control computer (31) to the master control computer (39), into a kinematics transformation (32), and transforming these forces to the master control interface (30) geometry so as to calculate the value of the force/torque that needs to be applied on each master motor (41) and thus to the human hand, then multiplication of the calculated value with a coefficient for scaling purposes,
Obtaining force error signals by means of subtracting, from the obtained reference signals, master external force signals applied on the master control interface (30) by the surgeon and estimated from each master motor (41),
Filtering the error signals through a band pass filter (40),
Subtracting a damping force signal generated by the damper (38) from the filtered signal so as to form reference forces for the master motors (41),
Adding the disturbance force values obtained from the disturbance estimator (35) to the reference forces, converting the total force value to current references, and transmitting thereof to the master DAQ card and motor drivers (44),
Achieving force control of the master control interface (30) by means of ensuring application of the reference forces by the master motors (41) through the current control of the master motors (41) by the DAQ card and motor drivers (44),
Conducting disturbance estimation on the motors (41) by means of the disturbance estimator (35) using the position measurements obtained from the master motors (41) and estimating, by means of the force estimation algorithm (36), the external forces applied onto the master motors (41),
Obtaining the speed signals of the motors (41) by means of processing the position measurement coming from the motors (41) using a speed estimator (37), and obtaining the damping forces by multiplying these signals by a coefficient of the damper (38),
Feeding of the position signals of the master motors (41) to a position controller (34) working in the robotic forceps control computer (31), as a control reference,
Finding the reference positions of the forceps motors (21) by sending the position signals of the master motors (41) to the kinematic transformation (32) that works on the robotic forceps control computer (31),
Forming the position error signals upon subtracting the measured positions of the forceps motors (21) from these reference positions, and forming a control force signals by inserting the error signals into the position controller (34),
Adding, to the created force signals, the disturbance forces coming from the disturbance estimator (35), and transforming thereof into current references for the motors and sending the same to the robotic forceps DAQ card and motor drivers (28),
Controlling the currents given to the motors (21) by the motor drivers and ensuring position control of the forceps motors (21) via motor current control,
To be used in the subsequent cycle, estimating the disturbance forces on the forceps motors (21) by means of the external force estimator (35), using the position measurement obtained from the forceps motors (21), and estimating the force applied on the forceps motors (21) by means of the external force estimator (36),
Sending force estimations to the master control computer (39) for conducting force control.

According to FIG. 10, the control and force estimation method (Architecture B) of the bilateral teleoperation controller working simultaneously in the master control computer (39) and the robotic forceps computer (31) comprises following process steps. The method foresees repetition of the steps several times per second, by the control system, as a loop.
Feeding of the force estimation signals, transmitted from the master control computer (39) to the robotic forceps control computer (31), into a kinematic transformation (32), and adaptation of these forces to the robotic forceps geometry so as to calculate the values of the forces that needs to be applied on each forceps motor (21) and thus the surgical environment, and then multiplication of the calculated value by a coefficient for scaling purposes,
Obtaining force error signals by means of subtracting, from the obtained signals, the forceps external force signals applied to the robotics forceps (29) by the surgeon environment and estimated by each robotics forceps motor (21),
Filtering these error signals through a band pass filter (40),
Subtracting damping force signals generated by the damper (38) from the filtered signals so as to form reference force signals for the forceps motors (21),
Adding the disturbance force values obtained from the disturbance estimator (35) to the reference forces, converting the total force values to motor current references, and transmitting thereof to the robotic forceps DAQ card and motor drivers (28),
Achieving force control of the robotic forceps (29) by means of ensuring application of the reference forces by the forceps motors (21) as a result of realization of current control of the forceps motors (21) by the DAQ card and drivers (28),
To be used in the subsequent cycle, estimating the disturbance on the forceps motors (21) by means of a disturbance estimator (35), using the position signal obtained from the forceps motors (21), and estimating the force applied on the motors (21) by means of the external force estimator (36),
Also to be used in the subsequent cycle, obtaining the speed signals of the forceps motors (21) by means of processing the position measurements coming from the motors (21) using a speed estimator (37), and obtaining the damping forces by multiplying these signals by a coefficient of the damper (38),
Submission of the position signals of the robotic forceps motors (21) to a position controller (34) working in the master control computer (39), as a control reference,
Finding the reference positions of the master (command interface) motors (41) by sending the position signals of the robotic forceps motors (21) to the kinematic transformation (32) that works on the master control computer (39),
Forming the position error signals upon subtracting the measured positions of the master motors (41) from these reference positions, and forming control force signals by inserting this error signal into the position controller (34),
Adding, to the created force signals, the disturbance effects coming from the disturbance estimator (35), and transforming thereof into current references and sending the same to the master DAQ card and motor drivers (44),
Ensuring position control of the master motors (41) via the control of the currents provided to the motors (41) by the motor drivers,
Conducting disturbance estimation on the motors (41) by means of the disturbance estimator (35) using the position measurements obtained from the master motors (41), to be used in the subsequent cycle, and estimating, by means of the external force estimator (36), the force applied onto the master motors (41),
Sending force estimations to the robotic forceps computer (31) for conducting force control.

According to FIG. 11, the disturbance estimator (35) and the external force estimator (36) employed simultaneously by both the robotic forceps control computer (31) and the master computer (39) and using both the Architecture A and Architecture B comprises the below given operations:
Entering the current signals sent to the motors (21, 41) into the motor models found in the master control computer (31) and robotic forceps control computer (39) (these models can be differential equations and transfer function models),
Obtaining model errors by means of subtracting, from the position signals obtained from the outputs of these models, the position signals measured from the motors (21, 41) with the help of the DAQ and Drivers (28, 44),
Calculation of the disturbance impact forces externally affecting the motors (21, 41) by inputting the obtained model errors into the motor inverse model (43),
Performing the motor disturbance estimation as a result of filtering of this signal by a band pass filter,
Calculating the trajectory of the motors and the robot using the motor current data and feeding the robot trajectory to inverse dynamics calculation (33),
Calculating, from the obtained results, the dynamic forces on the motors (21, 41) arising from the motion/trajectory following and estimating the external disturbance forces by subtracting the calculated dynamic force values from the total disturbances on the motors (21, 41),
Incorporating the external disturbance effects into kinematic transformation (32) algorithm and thus ensuring transformation/conversion of the external forces from the motor coordinates to the robot Cartesian coordinates when necessary.

The above mentioned force estimation and control methods are preferably used with the robotic forceps mechanism (29) as disclosed above in detail. However, it is also possible to run the said methods with different mechanism designs. In this context, it is important to ensure that the robotics forceps (29) design is capable of transforming the motor (21) motions to the desired degrees of freedom. Furthermore, it is important that when the robotic forceps mechanism (29) touches a surface while it is moving under the control algorithm, the reaction forces and motions formed as a result of the touch/contact are transmitted to the forceps motors (21) without significant loss, or in other words, the mechanism is backdrivable. Moreover, while one of the robots in the suggested control algorithm conducts position control, the other one makes force control. And this leads to a 2-channel communication between the robots. However, the 2-channel, 3-channel, 4-channel bilateral teleoperation algorithms known and applied in the literature can also be adapted to the system and used. The disturbance estimator used in the invention, together with the damping (38) and band pass filter (40), distinguish the method from the bilateral teleoperation algorithms used in the literature and enables superior performance. These are among the novel characteristics of the invention. However, if the stability of the system is not taken into account when there are no communication time delays, both robots can share their force and the position data and 4 channel architecture can be used in control algorithm. The most significant superiority of the bilateral teleoperation algorithm disclosed herein is the ability to maintain stability and high performance even when delays are present in the communication lines.

The system can be designed such that it would achieve the desired purposes even without the use of disturbance estimator (35) and the external force estimator (36) in the algorithm. Instead of external force estimation, force can be measured by positioning force sensors between each motor and the robot to which it is connected. If the disturbance estimations are not fed back to the motors in the control algorithm, then the forces estimated on the robots themselves are not required to be go through the force controller. However, in addition to the position controller, the external force estimated/measured on the robot where the position controller is running should also be negatively fed back. Disturbances arising from dynamic forces are supplied to the inverse dynamic estimator and fed back to system, and thus the errors due to dynamic effects may be minimized. Damping (38) and filter (40) are required for stability of the system. The position controller (34) can be a PID or equivalent controller.

In line with the above given information, the basic procedure steps of the method disclosed in the present invention are as follows;
Upon manual guidance of the master control interface (30) by the surgeon, master motor (41) data, changing as a result of the motion, is transmitted to the master control computer (39) via the master DAQ card,
The master control computer (39) sends commands to the master motors (41) by means of the master DAQ card by using the control and force estimation algorithms in order to ensure motion or immobility of the master control interface (30) in accordance with the master motors (41) information and the force/position information obtained from the robotic forceps computer (31),
The master control computer (39) sends the master force and position data coming from the master control interface (30) to the robotic forceps control computer (31),
The robotic forceps control computer (31) sends commands to the forceps motors (21) to perform the determined motion, by means of the robotic forceps DAQ card by using the control and force estimation algorithms in order to ensure the motion or immobility of the robotic forceps (29) in accordance with the position and/or force information coming from the master control computer (39) and the data coming from the forceps motors (21) by means of the robotic forceps DAQ card,
In line with the given command, orientation/guiding of the gripper section (A) so as to execute the desired motions upon transmission of the drive provided by the forceps motors (21) to the motion transmission rods (14), and then to the wrist motion mechanism (B) through the motion transmission rods (14),
transmission of the reaction forces generated as a result of contact when the gripper part (A) touches a surface, to the forceps motors (21) by means of the motion transmission rods (14),
Estimation of the force applied on the forceps motors (21) by control and force estimation algorithms via the robotic forceps control computer (31) or by force sensors when it is not possible to conduct force estimation via forceps motor (21),
Initiation of the next motion control cycle of master motors (41) upon transmission of the estimated forceps force data to the master control computer (39), and the reflection of the forces applied onto the surgical environment to the surgeon hand by means of the master motors (41).

In cases when force estimation is not directly sent to the master side; since the forceps position is sent and the forceps position is also controlled by force control, the operator will be able to feel the force feedback indirectly.

1. A backdrivable robotic forceps mechanism for use in robotic minimal invasive surgery, comprising a gripper part to perform gripping and cutting operations, a wrist motion mechanism allowing said gripper part to perform the desired wrist motions, a driving mechanism providing the drive required for movement of the said wrist mechanism, and an actuator transmission part providing motion transmission between the wrist motion mechanism and the drive mechanism and also transmitting the forces applied onto the wrist motion mechanism and the gripper to the drive mechanism, and it is characterized in that:
said driving mechanism comprises:
forceps motors allowing said wrist motion mechanism to perform rotating motions on x and y axes and back-and-forth motions on radial axis, and
shafts which are the movable parts of said forceps motors,
said actuator transmission section comprises motion transmission rods connected to the said wrist motion mechanism via revolute joints and only moving back-and-forth to transmit the motion coming from the forceps motors to the wrist motion mechanism, and at the same time ensuring transmission of the forces generated on the gripper part to the forceps motors, and
said wrist motion mechanism comprises:
interconnection pieces connecting the motion transmission rods to the wrist motion mechanism via revolute joints where the interconnection pieces intersect with both the lower connection parts and the motion transmission rods, and
connection parts, connected to the interconnection parts, the gripper section and each other via joints, and together converting the linear motion performed by the motion transmission rods into rotational motion around x and y axes and into thrusting motion in radial direction so as to enable the gripper part to perform the desired motion and ensuring transmission of the forces on the forceps to the forceps motors and vice versa.

2. The robotic forceps mechanism according to claim 1, and it is characterized in that the gripper section comprises:
a gripper performing the gripping/grasping and cutting operations;
a pin-slot mechanism the pin section of which is connected to the height fixing columns, the slot section of which is found on the gripper, which transforms the relative linear motion between the height fixing columns and the gripper into gripper opening and closing motion, and thus ensures obtaining jaw opening and closing motion by changing the radial position of the gripper with the coordinated movements of the forceps motors, and allowing transmission of the forces in the opening and closing motion axis of the gripper to the motors, or a mechanism like a 4-bar or a rack and pinion which would convert the relative linear motion into rotational jaw opening and closing motion,
a gripper base forming the ceiling of the wrist motion mechanism and the base of the gripper section, determining the general orientation/motion of the gripper, and providing relative motion of the slot with regard to the pin.

3. The robotic forceps mechanism according to claim 1, and it is characterized in that the wrist motion mechanism comprises:
upper connection pieces connected to the gripper base via revolute joints and ensuring movement of the gripper too, when the lower part of the mechanism is moved, and transmitting the forces generated at the gripper to the other parts of the mechanism,
primary mid-connection parts connected to the said upper connection parts, and the secondary mid-connection parts with mutually perpendicular revolute joints allowing the gripper to change its orientation via rotation on a single axis, and transferring the forces coming from the upper connection parts to the secondary mid-connection parts,
secondary mid-connection parts connected to the said primary mid-connection parts and the lower connection parts with mutually perpendicular revolute joints, allowing the gripper to change its orientation by means of rotating on a second axis that is perpendicular to the first rotation axis, and transferring the forces thereon to the lower connection parts,
lower connection parts connected to the said secondary mid-connection parts, and the interconnection pieces with mutually perpendicular revolute joints and providing transmission of the motion coming from the forceps motors to the gripper base and the forces coming from the gripper base to the forceps motors, by means of interconnection parts and motion transmission rods.

4. The robotic forceps mechanism according to claim 1, and it is characterized in that the wrist motion mechanism comprises:
a mechanism base serving as the base of the mechanism, to which the lower connecting parts are connected with revolute joints and all the static forces on the mechanism are transferred.

5. The robotic forceps mechanism according to claim 1, and it is characterized in that the wrist motion mechanism comprises:
height fixing columns composed of two or more columns connected to each other with spherical joints for preventing the back-and-forth motion of the pin section of the pin-slot mechanism found on the gripper, and thus providing relative motion of the gripper base and the slot section with respect to the pin section,
a height fixing column primary joint to which the height fixing columns are connected and which also allows the height fixing columns to change their orientation in one axis in accordance with the rotation of the gripper base/mechanism ceiling,
a height fixing column secondary joint on which said height fixing columns primary joint is connected, and which enables orientation change on another axis that is perpendicular to the primary joint rotation axis.

6. The robotic forceps mechanism according to claim 1, and it is characterized in that the wrist motion mechanism comprises a semi-rigid height rod that can perform similar function to rigid height fixing columns and joints, and capable of showing resistance to tension or compression in radial direction, but can change orientation without a joint, or a flexible height fixing string that may compress but can resist tension thus allowing force transmission to motors and force estimation and control in one direction at the gripper.

7. The robotic forceps mechanism according to claim 1, and it is characterized in that the actuator transmission section comprises: a primary base column and a secondary base column extending parallel to the said motion transmission rods along the actuator transmission part and supporting the mechanism base.

8. The robotic forceps mechanism according to claim 1, and it is characterized in that the actuator transmission section comprises: at least one shaft bearing set having openings where motion transmission rods can be inserted through and preventing out-of-axis motions of the motion transmission rods placed in these openings, and reducing the non axial loads thereon.

9. The robotic forceps mechanism according to claim 1, and it is characterized in that the driving mechanism comprises: connection rods, at one end of which the motion transmission rod and at the other end of which the shaft are connected so as to provide connection between the motion transmission rods and the forceps motors.

10. The robotic forceps mechanism according to claim 1, characterized in that a pedestal section on which the mechanism components are mounted.

11. The robotic forceps mechanism according to claim 10, characterized in that the pedestal section comprises: fixer members aligning the forceps motors relative to one another and fixing thereof to the base of the robotic forceps mechanism.

12. The robotic forceps mechanism according to claim 1, characterized in that said forceps motor is a linear motor or an actuator capable of converting rotational motion into linear motion via transmission components.

13. (canceled)

14. The robotic forceps mechanism according to claim 1, characterized in that it comprises a connection component ensuring connection of the mechanism to any robot arm in order to reach 7 degrees of freedom.

15. A robotic forceps control system for use in robotic minimal invasive surgery, comprising a robotic forceps mechanism to allow operation within patient body and a force feedback capable master control interface allowing remote control of the said robotic forceps mechanism by a surgeon, and it is characterized in that it comprises:
a wrist motion mechanism through which forceps motors change orientation of gripper section of the robotic forceps mechanism and enable gripper jaw opening-closing motion, and motion transmission rods transmitting the motion it receives from said forceps motors to the gripper section and also ensuring transmission of the forces generated at the gripper section to the forceps motors,
a robotic forceps control computer, which processes the master reference force and position information received from the control computer of the master control interface via its control and force estimation algorithms, and then transmitting control commands to the motors of the robotic forceps mechanism, and processes the forceps force and position information coming from the motors by means of the transmission rods, via its control algorithms, and transmitting thereof to the control computer of the master control interface,
a master control computer processing the force applied onto the master control interface by the surgeon and the position data by using the force estimation and control algorithms, and transmitting thereof to the robotic forceps control computer, and processing the forceps force and position data of the robotic forceps coming from the forceps control computer by using the force estimation and control algorithms, and thus affecting the master control interface motors, and thus allowing the surgeon to feel the force feedback from the forceps.

16. (canceled)

17. (canceled)

18. (canceled)

19. The robotic forceps control system according to claim 15, characterized in that said robotic forceps mechanism is the robotic forceps mechanism according to claim 1.

20. (canceled)

21. A robotic forceps control method applied by means of said robotic forceps control system according to claim 15, and it is characterized in that it comprises the operation steps of:
master motor data is transmitted to the master control computer via the master DAQ card upon manual guiding of the master control interface by the surgeon,
the master control computer sends command to the master motors by means of the master DAQ card by using the control and force estimation algorithms in order to ensure motion or immobility of the master control interface in accordance with the master motor data and the force/position data obtained from the robotic forceps control computer,
the master control computer sends the master force and position data measured or estimated from the master control interface to the robotic forceps control computer,
the robotic forceps control computer sends commands to the forceps motors to perform the determined motion, by means of the robotic forceps DAQ card by using the control and force estimation algorithms in order to ensure the motion or immobility of the robotic forceps in accordance with the position and/or force information coming from the master control computer and the data coming from the forceps motors by means of the robotic forceps DAQ card,
orientation/position of the gripper section is controlled so as to execute the desired motions upon transmission of the motion provided by the forceps motors to the motion transmission rods, and then to the wrist motion mechanism through the motion transmission rods in line with the given command,
transmission of the reaction forces and motion generated when the gripper part touches a surface, to the forceps motors by means of the motion transmission rods,
estimation of the forces coming from the gripper section and transmitted to the forceps motors by the control and force estimation algorithms running on the robotic forceps control computer or by the force sensors when it is not possible to conduct force estimation via forceps motor,
initiation of the next motion control cycle of master motors upon transmission of the estimated forceps force and position data to the master control computer, and the reflection of the environmental reaction forces on the forceps to the surgeon hand by the master motors.

22. The robotic forceps control method according to claim 21, characterized in that the control and force estimation method applied by the master control computer according to a version of the forceps mechanism running under position control comprises the operation steps of:
feeding the force estimation signals, transmitted from the robotic forceps control computer to the master control computer, into a kinematics transformation, and transforming these forces according to the master control interface geometry so as to obtain the values of the force/torque that needs to be applied on each master motor and thus to the surgeon hand, and then multiplying the calculated values with a coefficient for scaling purposes,
obtaining force error signals by means of subtracting, from the obtained reference signals, external force signals from the master control interface as a result of interaction with the surgeon and estimated from each master motor,
filtering these error signals by passing through a band pass filter,
producing reference forces for the motors by subtracting the damping force signals generated by the damping from the filtered signals,
adding the disturbance estimate values obtained from the disturbance estimator to the reference forces, converting these total force values to a current reference, and transmitting thereof to the master DAQ card and drivers,
achieving force control of the master control interface by means of application of the reference forces by the master motors via current control of the master motors by the DAQ card and motor drivers,
to be used in the next cycle, conducting disturbance estimation on the motors by means of the disturbance estimator using the position measurement obtained from the motors, and estimating the external force applied onto the master motors by means of the force estimation algorithm,
also to be used in the next cycle, obtaining the speed signals of the motors by means of processing the position measurements coming from the motors using a speed estimator, and obtaining the damping force by multiplying these signals by a coefficient of the damping,
feeding the position signals of the motors to a position controller working in the robotic forceps control computer, as a control reference,
finding the reference positions of the forceps motors by sending the position signals of the motors to the kinematic transformation that works on the robotic forceps control computer,
forming the position error signals upon subtracting the measured positions of the forceps motors from these reference positions, and forming control force signals by inserting these error signals into the position controller,
adding the disturbance estimations coming from the disturbance estimator to the created force signals and transforming thereof into current references and sending the same to the robotic forceps DAQ card and motor drivers,
controlling the currents given to the motors by the motor drivers, and thus achieving position control of the forceps motors indirectly,
to be used in the subsequent cycle, estimating the disturbance on the forceps motors by means of the external force estimator, using the position measurements obtained from the forceps motors, and estimating the forces applied on the forceps motors by means of the external force estimator,
sending force estimations to the master control computer for conducting force control.

23. The robotic forceps control method according to claim 21, characterized in that the control and force estimation method applied by the robotic forceps control computer according to a version of the forceps mechanism running under force control comprises the operation steps of:
feeding of the force estimation signal, transmitted from the master control computer to the robotic forceps control computer, into a kinematic transformation and transformation of this force according to the robotic forceps geometry so as to calculate the value of the force that needs to be applied on each forceps motor and thus to the surgical environment and then multiplication of the calculated value by a coefficient for scaling purposes,
obtaining force error signals by means of subtracting the external forces applied to the robotics forceps and thus to forceps motors by the surgical environment and estimated from robotic forceps motor measurements,
filtering these error signals by feeding through a band pass filter,
producing reference forces for the motors by subtracting the damping force signals generated by the damping from the filtered signals,
adding the disturbance force values obtained from the disturbance estimator to the reference forces, converting these total force values to a current reference, and transmitting thereof to the robotic forceps DAQ card and motor drivers,
achieving force control of the robotic forceps by means of ensuring application of the reference forces by the forceps motors through the current control of the forceps motors by the DAQ card and drivers,
to be used in the subsequent cycle, estimating the disturbances on the forceps motors by means of the disturbance estimator, using the position signals obtained from the robotic forceps motors, and estimating the forces applied on the motors by means of the external force estimator,
also to be used in the subsequent cycle, obtaining the speed signals of the motors by means of processing the position measurements coming from the forceps motors using a speed estimator, and obtaining the damping forces by multiplying these signals with a coefficient by the damping,
submitting the position signals of the robotic forceps motors to a position controller working in the master control computer, as a control reference,
finding the reference positions of the master motors by sending the position signals of the robotic forceps motors to a kinematic transformation that works on the master control computer,
forming the position error signals upon subtracting the measured positions of the master motors from these reference positions and forming control force signals by inserting these error signals into a position controller,
adding the disturbance estimations coming from the disturbance estimator to the created force signals and transforming thereof into a current reference and sending the same to the master DAQ card and motor drivers,
controlling the currents given to the motors by the motor drivers, and thus ensuring position control of the master motors indirectly,
conducting disturbance estimation on the motors by means of the disturbance estimator using the position measurements obtained from the master motors, to be used in the subsequent cycle, and estimating the force applied onto the master motors by means of the external force estimator,
sending force estimations to the robotic forceps computer for conducting force control.

24. The robotic forceps control method according to claim 22, and it is characterized in that the following operations are conducted by means of the disturbance estimator:
entering the current signals sent to the motors into the motor models found in the master control computer and robotic forceps control computer,
obtaining model errors by means of subtracting the position signals measured from the motors with the help of the DAQ card and motor drivers from the position signals obtained from the outputs of these models,
calculating the disturbance forces externally affecting the motors by inputting the obtained model errors into the motor inverse model, and
performing the motor disturbance estimation as a result of filtering of this signal by a band pass filter.

25. The robotic forceps control method according to claim 22, and it is characterized in that the following operations are conducted by means of the external force estimator:
calculating the trajectory of the robot using the motor current data and feeding the robot trajectory data to the inverse dynamics calculation,
calculating the dynamic forces arising from the motion over the robot motors from the determined robot trajectory and inverse dynamics calculation, and estimating the external disturbance force by subtracting the calculated value from the total disturbance on the motor,
incorporating the external disturbance effect into kinematic transformation algorithm and thus ensuring transformation/conversion of the external forces from the motor coordinates to the robot Cartesian coordinates.
